(19) United States
(12) Patent Application Publication
(10) Pub. No.: US 2009/0074839 A1
(43) Pub. Date: Mar. 19, 2009

(54) Pharmaceutical Compositions Primarily for the Treatment and Prevention of Genitourinary Infections and their Extragenital Complications

(76) Inventor: Marton Milankovits, Budapest (HU)

Correspondence Address: Haugen Law Firm PLLP, 121 South Eighth Street, TCF Tower 1130, Minneapolis, MN, 55402, US

(21) Appl. No.: 12/274,535

(22) Filed: Nov. 20, 2008

Related U.S. Application Data
(63) Continuation-in-part of application No. 11/076,194, filed on Mar. 9, 2005, now abandoned.
Application No. 11/076,194 is a continuation-in-part of application No. 10/192,345, filed on Jul. 10, 2002, now abandoned.
Application No. 10/192,345 is a continuation-in-part of application No. 08/776,273, filed on Jan. 22, 1997, now Pat. No. 6,432,935.

(51) Int. Cl. A61F 13/20; A61K 33/22; A61K 31/4025; A61K 31/4164

(52) U.S. Cl. 424/431; 424/658; 514/266.23; 514/422; 514/398

(57) ABSTRACT

Compositions having synergistic effective amounts of one or more antibacterial agents, a nitroimidazole, and/or a sulfonamide or a molecule or compound having folic acid antagonist effect and nitroimidazole substitution in addition, with or without an antifungal agent effective against a Candida species. The compositions are particularly useful in the treatment of genitourinary infections and their extragenital complications. Vaccination against pathogen microbes of the vagina provides a stronger and longer immune response, then the infection.

### CROSS-REFERENCE TO RELATED APPLICATION

This application is a continuation-in-part of U.S. application Ser. No. 11/076,194, filed Mar. 9, 2005 and entitled “Pharmaceutical Compositions Primarily for the Treatment and Prevention of Genitourinary Infections and Their Extragenital Complications”, which is a continuation-in-part of U.S. application Ser. No. 10/192,345, filed Jul. 10, 2002, and entitled “PHARMACEUTICAL COMPOSITIONS PRIMARILY FOR THE TREATMENT OF GENITO-URINARY INFECTIONS”, which application itself is a continuation-in-part of U.S. application Ser. No. 08/776,273, filed Jan. 22, 1997, and entitled “VAGINAL SUPPOSITORIES AND NOVEL PHARMACEUTICAL COMPOSITIONS”, the contents of which are herein incorporated in their entirety.

### FIELD OF THE INVENTION

This invention relates to novel compositions and their uses in treatments as suppositories, especially vaginal suppositories, ointments, vaginal drops and talc powders, and painting solutions, or any form of the compositions useful for systemic treatment. Substitution of a nitroimidazole (made in the amide NH2 group of Sulfanilamide, or in molecules or compounds having anti-folic acid effectiveness) yields new compounds having combined effectiveness. Vaccination can provide stronger and longer immune response than the natural one (polyvalent serum).

### BACKGROUND OF THE INVENTION

A variety of vaginal suppositories are currently commercially available for the treatment of various maladies. The attending physician ordinarily decides which composition is best suited to the patient's needs following physical examination.

For example, for the treatment of vaginal mycosis, Canesten (active ingredient is clotrimazol; bis-phenyl-(2-chlorophenyl)-1-(imidazolyl)-methane; and Pimafucin (the active ingredient is natamycin-primaricin) are most commonly used. For fungal and protozoan infection, Klion D (the active ingredient is metronidazole; 1-(2′-hydroxiethyl)-2-methyl-5-nitroimidazol and myconasol-nitrate) is used. For protozoan infection, Klion vaginal suppository (active ingredient is metronidazole) is commonly used.

Certain compositions exert their effects through the disinfective action of iodine. These include Betadine (iodine is released from the carrier). Other vaginal suppositories feed the natural flora of the vagina. These include Genia 92 nutrients, e.g.: folic acid, lactic acid, lactose, and lactamine.

A common disadvantage of the above compositions is that none of them makes possible the combination of the effects of (i) bactericide (for aerobe and anaerobe bacteria), involving anti-Mobiluncus and anti-Gardnerella, (ii) fungicide, and (iii) anti protozoa simultaneously. Moreover, they have no antiviral effect at all.

### SUMMARY OF THE INVENTION

The invention relates to compositions which make possible the attack of pathogens from different directions, which simultaneously aid in the body's antiviral struggle. The invention facilitates rapid and simple selection of the safest and most useful compositions.

The basis of the invention is the discovery that a unique combination of active ingredients has numerous advantages over the art. In a preferred embodiment, the composition of the present invention includes an antibacterial agent, an antifungal agent effective against a Candida species, and a nitroimidazole, wherein the antibacterial agent, the antifungal agent, and the nitroimidazole are present in the composition in synergistic effective amounts. Preferably, the compositions of the present invention further include a pharmaceutically acceptable carrier.

The basis of the discovery according to the invention is that the effect of the antibacterial agent ingredient is unexpectedly intensified by the other active components of the present composition. For example, the antibacterial effect, particularly of chloramphenicol and sulfonamide, against Chlamydia trachomatis is greatly increased by the present compositions. Additionally, an increased inhibitory effect of the antibacterial agent, particularly chloramphenicol and nitroimidazole, particularly metronidazole, components against anaerobe pathogens (e.g. B. fragilis) was observed when utilized in compositions of the present invention. The antibacterial effect of the sulfonamide and/or nitroimidazole also unexpectedly potentiated antibiotics generally against each pathogenic bacterium, in the antifungal protection provided by the antifungal agent. The antifungal agent or agents play a minor role in the synergistic effect.

The unexpected increased effectiveness of the components of the present composition when present in synergistic effective amounts has been observed when utilizing the three ingredients listed above. A further aspect and embodiment of the present invention provides for the inclusion of a sulfonamide in addition to another antibacterial agent, the antifungal agent, and the nitroimidazole, when present in a composition in synergistic effective amounts. As such, another embodiment of the present invention comprises an antibacterial agent, a sulfonamide, an antifungal agent effective against a Candida species, and a nitroimidazole, wherein each of these components are present in a composition in synergistic effective amounts. Preferably, the composition further includes a pharmaceutically acceptable carrier for obtaining a suitably deliverable medication to a patient.

It has been observed through experimental data that the antibacterial effect of sulfonamide standing alone is greatly enhanced when coupled with the other ingredients of the present compositions. Therefore, such an unexpected increased effectiveness of sulfonamide when incorporated into the compositions of the present invention forms an additional embodiment and aspect of the present invention.

An additional aspect and embodiment of the present invention further supports the unexpected increased effectiveness of sulfonamide when combined with other active ingredients. A particular example which has proven unexpectedly effective against bacterial vaginosis is the combination of a sulfonamide and a nitroimidazole.

Thus, the treatment spectrum is broader and the effect of the combination is much stronger than would be expected from its individual components, while simultaneously decreasing the necessary dosage for treatment compared to the individual active ingredients. This results in a decrease of the possible side effects while using the present combination of elements. Another significant advantage of the solution according to the invention is that drug resistance does not occur. The main role in the synergistic effect is played in the compositions of the invention by the nitroimidazole and/or sulfonamide component(s), probably causing apoptosis of the involved eukaryotic cells and by killing most of the prokaryotic cells. (Apoptosis-like additional effect). The level of the mechanism of action is the same in the case of a nitroimidazole or a sulfonamide. Both of them act on transcriptional level. The unity of the invention more explicitly is the following: The basis of the invention is the discovery that the nitroimidazoles and sulfonamides potentiate the effectiveness of each other and/or the effectiveness of other antimicrobial agents. Both the nitroimidazoles and the sulfonamides impair the intracellular metabolism and act on DNA level too. Each of them weakens the target cells (The target cells are prokaryotic cells and unhealthy eukaryotic cells). Thus, the second antimicrobial agent or agents of the compositions of the invention is/are unexpectedly effective on the weakened target cells (the sulfonamide component can be replaced by a combination of a sulfonamide and trimethoprim, because their combined effectiveness on cell metabolism is well known).

An additional aspect and embodiment of the present invention is the group of new molecules containing the radicals of the components mentioned above. The antifungal component(s) effective against Candida of the present invention further contribute to the unexpected increased efficacy of the compositions. Problematic super-infections with Candida occur very frequently and this is the second important reason why necessary combined antifungal treatment and the antifungal components should be effective against Candida. Similarly to the combined antibacterial treatment, the combined antifungal treatment is much more effective than the monotherapy. The compositions of the present invention are equally effective in the cases of systemic and local treatments and shorten the duration of the antigen stimulus which may resulted in the need of vaccination. A treatment, however, without a full “sterilizing” dose of a suitable substance results in the development of “serum resistant strains” (Paul Ehrlich, Partial cell functions, Nobel Lecture, Dec. 11, 1908).

### DETAILED DESCRIPTION OF THE INVENTION

The present invention relates to novel compositions having synergistic effective amounts of one or more antibacterial agents, a nitroimidazole, and an antifungal agent effective against a Candida species. The antibacterial agents used in the present invention are also commonly referred to as antibiotics. The present invention contemplates the use of any antibiotic as defined by Martindale—The Extra Pharmacopoeia, 29th Ed, London, The Pharmaceutical Press, 1989 (hereinafter “MARTINDALE”). As stated in MATINDALE, page 94:
Antibiotics have traditionally been divided into bacteriostatic antibiotics which reversibly inhibit the growth of susceptible microorganisms and bactericidal antibiotics which kill the organisms in vitro. Given in high therapeutic doses, the aminoglycosides, cephalosporins, penicillins, and polymyxins are generally bactericidal by this criterion whereas chloramphenicol, erythromycin, the sulfonamides, and the tetracyclines are usually bacteriostatic. However, an antibiotic which is bactericidal in a certain concentration may become bacteriostatic at lower concentrations.

In addition, antibiotics are classified into five classes based on chemical structure and mechanism of action in Chapter 43 of Goodman & Gilman's The Pharmacological Basis of Therapeutics, 9th Ed. (McGraw-Hill, 1996) (hereinafter “GOODMAN-GILMAN”). Such classes are set forth in Chapter 43, pages 1029-1030 of GOODMAN-GILMAN. As described therein, the individual members of each class are interchangeable with one another since they act on the target microorganism in the same way, and in most cases are also chemically interrelated with one another.

Experimental data set forth in the examples detailed below indicate that antibiotics belonging to each of the five classes defined in Chapters 43-48 of GOODMAN-GILMAN are effective and preferred for use in compositions of the present invention.

In some embodiments of the present invention, it is desired to utilize a sulfonamide for the particularly desirable antibiotic effect thereof when used in combination with the other components of the composition of the present invention. Any sulfonamide may be utilized in the composition of the present invention as all sulfonamides are structural analogs and competitive antagonists of para-aminobenzoic acid (see GOODMAN-GILMAN page 1058). Though all sulfonamides are interchangeable as applied to the composition of the present invention, a particularly preferred sulfonamide is sulfadimidin. In applications where a patient has an allergy to sulfonamides, it is preferred to formulate the composition of the present invention without inclusion of a sulfonamide.

The antifungal agents utilized in the compositions of the present invention are preferably effective against Candida species (Candida albicans, Candida stelloidea, etc). Antifungal agents are divided into groups in GOODMAN-GILMAN, Chapter 49, pages 1175-1190, defining antifungal agents effective against Candida species which act only topically, and those agents effective against Candida species both systemically and topically. The antifungal agents effective against Candida species which act both systemically and topically are divided into the following subclasses: polyenes, azoles, and pyrimidines. According to GOODMAN-GILMAN, Chapter 49, members of each subclass are interchangeable, in that each member of a respective subclass exhibits similar chemical properties and functionalities. Characteristic representatives of the polyenes are natamycin and nystatin. A characteristic representative of the azole subclass is clotrimazole. A characteristic representative of the pyrimidine subclass is flucitozine.

Another subclass of antifungal agents effective against Candida species are those antifungal agents which act only topically. Characteristic representatives of this subclass are ciclopirox olamine, naftifine, terbinafine, and haloprogin. Though the antifungal agents selected for use in the compositions of the present invention are preferably effective against Candida species, it is not necessary that such antifungal agents be effective solely against such Candida species. For example, particular selected antifungal agents of the present invention may be effective against a multiplicity of fungi.

The nitroimidazole component of the compositions of the present invention further contribute to the unexpected increased efficacy of each component of the compositions. The mechanism of action of the nitroimidazoles is set forth in GOODMAN-GILMAN, page 996 as reflecting a selective toxicity to anaerobic or microaerophilic microorganisms. As such, the members of the nitroimidazole group share a common functionality and are so closely related in structure so as to be interchangeable (see GOODMAN-GILMAN). Characteristic representatives of the nitroimidazole group are metronidazole and tinidazole.

In one embodiment, the composition of the present invention includes:

| Component | Amount |
|---|---|
| a) Antibacterial Agent | 0.04-0.30 g |
| b) Antifungal Agent | 0.025-0.30 g |
| c) Nitroimidazole | 0.10-0.45 g |

A pharmaceutically acceptable carrier for the compositions of the present invention is polyethylene-glycol, but other suitable carriers may also be employed. The amount of polyaethylene-glycol supplements the combination of the active ingredients to the necessary amount in case of a 10 unit package.

One aspect of the present invention is the unexpected increased efficacy of the presently disclosed components when utilized in the compositions of the present invention, as contrasted with the minimal effectiveness of such components standing alone. Commercially available medications containing only one component of the compositions of the present invention have been found to be ineffective, even after extended treatment periods or administration of large doses. Monotherapy, which is the treatment with a single active ingredient, is ineffective, in that a relapse rate of vaginal bacterial vaginosis is about 30-35% of the patients receiving such monotherapy, even over a treatment duration of up to 20 days.

Through clinical trials, Applicant has discovered an unexpected synergy of effectiveness in compositions consisting of the three active ingredients referred to above.

The combination of elements used in the compositions of the present invention are more effective and qualitatively different from the separate administration of the individual components. Such compositions may be contained and administered in the form of vaginal suppositories, ointments, vaginal drops, talc powders, and painting solutions. Administration of the present compositions has resulted in complete recovery in cases when recovery could not be reached by the separate administration of the components. The compositions are useful in a variety of applications and treatments, including the following:

Prophylactic Use. Use of the present compositions prevent infection from infected swimming-pool water or sexual activity. The compositions are indispensable prior to gynecological operations (especially utero-vaginal interventions) as a prophylactic suppository.

Treatment of Infection. For infections, use of a preparation containing a composition of the present invention results in absolute recovery in 90% of the cases.

Since systemic treatment is not needed, a smaller dose is administered. Cessation of treatment results in side effects disappearing (for local treatments there were no side effects observed). Resistance of the pathogens against the components used is also obviated because in local treatment the relatively small amount of preparation applied absolutely kills the pathogens.

Treatment of Chronic Vaginitis and the Alterations of the Cervix and of the Uterus. Presently, techniques such as laser surgery, conventional surgery, cryocoagulation or electro-cauterization are used for the treatment of the cervicalization (ectopium), and the positive epithelial differences (such as the acetic acid positive epithelium and the decrease of the iodine positivity) and P3 or repeatedly P3 cytological findings.

The compositions of the present invention promote the spontaneous healing of the bleeding, inflamed portion of the uterus. As a result, the above-described invasive conventional treatments become unnecessary. Constant inflammation plays a decisive role in the formation of cancer of the cervix of the uterus. Application of the compositions according to the invention greatly diminishes the risk of the formation of the cancer of the cervix of the uterus, by stopping the inflammation. Nitroimidazole has certain anticancer effects, which are observed during the radiation treatment of the tumors, where it increases the efficacy of the radiation treatment. In addition, it has been postulated that sulfonamides display anti-tumor characteristics as well (see Supuran C T et al. Carbonic Anhidrase Inhibitors: Sulfonamides as Antitumor Agents? Biorg. Med. Chem., March, 2001, 9(13):703-714).

Following the treatment with the present compositions, the laboratory findings improved from P3 cytological results to P2 or P1 without exception and the epithelium became normal kolposcopically.

Treatment of Complications of Ascending Infections. Ascending infections such as Cystitis, PID, etc. are typically caused by the same microbes as are in the vagina. The systemic application of the present combinations unexpectedly shorten the usual duration of treatment.

Later complications of the inflammation (infection) can be vascular damage anywhere in the body (e.g. atherosclerosis and the connected diseases (see Ross, R. Mechanism of disease: Atherosclerosis—an inflammatory disease. N. Engl. J. Med. 1999, 2:115-126.), neovascularisation, etc.).

Chronic inflammation has been shown to be an important risk factor for a variety of epithelial cancers, including those of the esophagus, stomach, pancreas, liver, biliary tract, colon, vulva and bladder (see Stephen E. Hawes, Nancy B. Kiviat. (editorials) Are Genital Infections and Inflammation Cofactors in the Pathogenesis of Invasive Cervical Cancer?, Journal of the National Cancer Institute 2002, 94:1592-1593).

Antiviral Effect. The present combination acts against viral infections, possibly by killing all non viral pathogens, enhancing the immunocapacity of the body against the viruses.

In those cases when, because of inflammation and vaginal discharge, the treatment of the Condyloma acuminatum (caused by the human papilloma virus) failed, local treatment using the present composition proved to be permanently successful.

Moreover, it was observed that patients with frequently reoccurring herpes genitalis, became permanently free of symptoms following the treatment with the preparation of the present invention.

The preparation according to the invention can also be administered to pregnant women to prevent adverse pregnancy outcome (and probably toxemias of pregnancy too). In such applications, erythromycin is the preferred antibacterial agent component.

Application in the Veterinary Medicine. The treatment of kolpitis in female dogs by the present combinations proved similarly effective as compared to human treatment.

Treatment of the inflamed glandula Bartholini by drainage and injected solution of a composition of the invention instead of simple incision or marsupialization. Other similar applications are also possible inside the body.

Combined (local and systemic) treatment: (a) In pregnancy to prevent intrauterine Chlamydia trachomatis infection causing pneumonia or perhaps blindness (see Milánkovits, Márton: The Possible Role Of Non Trachoma Chlamydia Trachomatis Serovars In Intrauterine Blindness, 12th Congress of the European Association of Gynecologists & Obstetricians, 25-28 Jun., 1997, Trinity College, Dublin, Ireland.); and (b) In cases of infections of sportswomen (mainly carriers) to improve their physical abilities and fitness.

The compositions according to the invention were tested on 300-400 cases, with 300-400 controls. In all cases, the full microbiological examination included Chlamydia trachomatis and the Mycoplasmas.

Preparations of the compositions according to the invention can be carried out by methods known in the art for the preparation of such compositions.

The following experimental results demonstrate the unexpected efficacy of the compositions of the present invention, and set forth exemplary compositions indicative of the many possible species combinations useful in the treatment of various maladies. The examples will serve to further typify the nature of the invention, but should not be construed as a limitation on the scope thereof, which is defined solely by the appended claims.

### EXAMPLE 1

309 Patients
Seven days treatment

| | |
|---|---|
| Chloramphenicol: | 0.10 g |
| Sulfadimidin: | 0.10 g |
| Nystatin: | 0.10 g |
| Metronidazole: | 0.40 g |
| Massa | 2.00 g |
| Polyoxaethenum: | |

Completely treated: 306
Remained the same: 3
Worsened: 0

### EXAMPLE 2

90 Patients
Seven days treatment

| | |
|---|---|
| Ciprofloxacin: | 0.04 g |
| Sulfadimidin: | 0.10 g |
| Nystatin: | 0.10 g |
| Metronidazole: | 0.40 g |
| Massa | 2.00 g |
| Polyoxaethenum: | |

Completely treated: 89
Remained the same: 1
Worsened: 0

### EXAMPLE 3

77 Patients
Seven days treatment

| | |
|---|---|
| Ampicillin: | 0.20 g |
| Sulfadimidin: | 0.10 g |
| Nystatin: | 0.10 g |
| Metronidazole: | 0.40 g |
| Massa | 2.00 g |
| Polyoxaethenum: | |

Completely treated: 75
Remained the same: 2
Worsened: 0

### EXAMPLE 4

60 Patients
Seven days treatment

| | |
|---|---|
| Sulfadimidin: | 0.10 g |
| Nystatin: | 0.10 g |
| Metronidazole: | 0.40 g |
| Massa | 2.00 g |
| Polyoxaethenum: | |

Completely treated: 52
Remained the same: 8
Worsened: 0

### EXAMPLE 5

19 Patients
Seven days treatment

| | |
|---|---|
| Neomycin: | 0.10 g |
| Sulfadimidin: | 0.10 g |
| Nystatin: | 0.10 g |
| Metronidazole: | 0.40 g |
| Massa | 2.00 g |
| Polyoxaethenum: | |

Completely treated: 18
Remained the same: 1
Worsened: 0

### EXAMPLE 6

10 Patients
Seven days treatment

| | |
|---|---|
| Polymixin: | 0.05 g |
| Sulfadimidin: | 0.10 g |
| Nystatin: | 0.10 g |
| Metronidazole: | 0.40 g |
| Massa | 2.00 g |
| Polyoxaethenum: | |

Completely treated: 9
Remained the same: 1
Worsened: 0

### EXAMPLE 7

12 Patients
Seven days treatment

| | |
|---|---|
| Semicillin: | 0.20 g |
| Sulfadimidin: | 0.10 g |
| Nystatin: | 0.10 g |
| Tinidazole: | 0.40 g |
| Massa | 2.00 g |
| Polyoxaethenum: | |

Completely treated: 11
Remained the same: 1
Worsened: 0

### EXAMPLE 8

10 Patients
Seven days treatment

| | |
|---|---|
| Semicillin: | 0.20 g |
| Sulfadimidin: | 0.10 g |
| Clotrimazole: | 0.10 g |
| Metronidazole: | 0.40 g |
| Massa | 2.00 g |
| Polyoxaethenum: | |

Completely treated: 9
Remained the same: 1
Worsened: 0

### EXAMPLE 9

10 Patients
Seven days treatment

| | |
|---|---|
| Semicillin: | 0.20 g |
| Sulfadimidin: | 0.10 g |
| Natamycin: | 0.30 g |
| Metronidazole: | 0.40 g |
| Massa | 2.00 g |
| Polyoxaethenum: | |

Completely treated: 9
Remained the same: 1
Worsened: 0

### EXAMPLE 10

10 Patients
Seven days treatment

| | |
|---|---|
| Chloramphenicol: | 0.10 g |
| Nystatin: | 0.10 g |
| Metronidazole: | 0.40 g |
| Massa | 2.00 g |
| Polyoxaethenum: | |

Completely treated: 8
Remained the same: 2
Worsened: 0

### EXAMPLE 11

10 Patients
Seven days treatment

| | |
|---|---|
| Semicillin: | 0.20 g |
| Natamycin: | 0.30 g |
| Metronidazole: | 0.40 g |
| Massa | 2.00 g |
| Polyoxaethenum: | |

Completely treated: 7
Remained the same: 3
Worsened: 0

### EXAMPLE 12

8 Patients
Seven days treatment

| | |
|---|---|
| Chloramphenicol: | 0.10 g |
| Sulfadimidin: | 0.10 g |
| Nystatin: | 0.10 g |
| Metronidazole: | 0.10 g |
| Massa | 2.00 g |
| Polyoxaethenum: | |

Completely treated: 7
Remained the same: 1
Worsened: 0

### EXAMPLE 13

Systemic administration (per os)
9 Patients
Seven days treatment

| | |
|---|---|
| Ciprofloxacin: | 750 mg/day (500 mg + 250 mg) |
| Ketoconazole: | 400 mg/day (200 mg + 200 mg) |
| Tinidazole: | 1000 mg/day (500 mg + 500 mg) |

Completely treated: 8
Remained the same: 1
Worsened: 0

The above examples demonstrate the interchangeability of specific species within the respective genera antibacterial agent, nitroimidazole, and antifungal agent effective against a Candida species, while maintaining the efficacy of the various compositions of the present invention. Examples 10 and 11 illustrate the high level of effectiveness of even three component compositions of the present invention. Such effective experimental results are particularly unexpected when contrasted with the treatment efficacy using a single active ingredient. Such monotherapy treatments result in less than 60% complete recovery (e.g. for bacterial vaginosis).

The compositions demonstrate significantly reduced treatment periods compared to application of these components separately. Previous treatment of the patients comprising the above test groups had failed with standard local administration of:

| Agent | Duration of Treatment | |
|---|---|---|
| Klion (metronidazole) | 10 | days |
| Klion D | 10 | days |
| Pimafucin (natamycin) | 20 | days |
| Canesten (clotrimazol) | 8-10 | days |

For example, it has been found that no benefit is realized through repeated 2000 mg doses of metronidazole administered alone (see Carey J C, Klebanoff M A, et al. Metronidazole to Prevent Pre-term Delivery in Pregnant Women with Asymptomatic Bacterial Vaginosis, New England Journal of Medicine 2000; 342:534-40). In a further aspect of the present invention, it has been found that the respective effectiveness of a nitroimidazole and a sulfonamide when utilized alone is enhanced when combined. As such, an unexpected success rate of over 60% against bacterial vaginosis is achieved through the combination of a sulfonamide and a nitroimidazole. In the compositions of the invention the sulfonamides or nitroimidazoles are unexpectedly strongly effective against most of the prokaryotic cells and the unhealthy mammalian (eukaryotic) cells too by causing their early death. (Unhealthy means infected cells and tumor cells). The TP53 gene encodes P53. One of its guardian functions is to stop cells from replicating damaged DNA. Normal cells with damaged DNA arrest at a checkpoint at the G1/S stage of the cell cycle until the damage is repaired, but unhealthy cells do not. Probably related to not normal “unhealthy cells” is a crucial role of p53 in programmed cell death (apoptosis). In the compositions of the present invention the (a) nitroimidazoles (mutagenic effects) and (b) sulfonamides (antifolates-teratogen effects) caused damage in DNA respectively, and therefore unexpectedly increase the effectiveness of the other ingredient or ingredients.

(a) Early work established that metronidazole inhibits DNA synthesis in T. vaginalis and Clostridium bifermentans and causes degradation of existing DNA in the latter microorganism. These findings are consistent with the antimicrobial and mutagenic effects of metronidazole. Once the drug has diffused into the cells, the nitro group accepts electrons from electron-transport proteins with sufficiently low negative redox potentials, e.g. flavoprotein in mammalian cells and ferredoxins or their equivalent in protozoa and bacteria (see GOODMAN-GILMAN page 996).

(b) Sulfonamides are structural analogs and competitive antagonists of para-aminobenzoic acid (PABA), and thus prevent normal utilization of PABA for the synthesis of folic acid. One of the most active antibacterial agent that exerts a synergistic effect when used with a sulfonamide is trimethoprim. The simultaneous administration of these agents thus introduces sequential blocks in the pathway of the synthesis of tetrahydrofolate from precursor molecules (see GOODMAN-GILMAN page 1058). Sulfonamides do not affect healthy mammalian cells. Antifolates occupy a special place in antineoplastic chemotherapy, e.g. Methotrexate—its toxicity includes teratogenesis.

Examples in the present application supporting (a) and (b) are as follows:

(1) Precancerous cells died: Following the treatment with the present compositions, the cytological findings of Papanicolau smears improved from P3 to P2 or P1. The two cases of P4 improved to P2 and P1 respectively. The precancerous or cancerous cells disappeared. They died because the mutagenic effect can damage the DNA of the cells but can not repair DNA and thus cure the cells; and

(2) Infected cells of Condyloma acuminatum died (the infection caused by the human papilloma virus): Probably because of the early death of the host cells, the formation of mature infectious progeny virus is not possible, perhaps the steps of the viral replicative cycle could not be successfully completed.

It has been observed through experimental data that the antibacterial or antitumor effect of sulfonamide or nitroimidazole standing alone is greatly enhanced when coupled with the other antibacterial ingredient or ingredients of the present compositions. Therefore, such an unexpected increased effectiveness of sulfonamide or nitroimidazole when incorporated into the compositions of the present invention forms an additional embodiment and aspect of the present invention.

Sulfonamides are structural analogs and so competitive antagonists of para-aminobenzoic acid (PABA): H₂N—COOH. They prevent the synthesis of folic acid and this prevention results in folic acid antagonist effect. The term sulfonamide is employed herein as a generic name for derivates of para-aminobenzenesulfonamide (sulfanilamide): H₂N⁴—SO₂N¹H₂ (e.g. sulfamethoxazole: H₂N—SO₂NH—)

The minimal structural prerequisites for antibacterial action are all embodied in sulfanilamide itself. The SO₂NH group is not essential as such, but the important feature is that the sulfur is directly linked to the benzene ring. The para-NH₂ group (the N of which has been designated as N4) is essential and can be replaced only by such radicals as can be converted in vivo to a free amino group. Substitutions made in the amide NH₂ group (the N of which has been designated as N1) have variable effects on antibacterial activity of the molecule. Substitution of heterocyclic aromatic nuclei at N yields highly potent compounds, (e.g. sulfamethoxazole).

An additional aspect and embodiment of the present invention is nitroimidazole substitution made in the amide NH₂ group of sulfanilamide or the same substitution(s) made in the single ring of the lipid soluble antifolate trimetrexate. The effectiveness of the antifolates such as the p-Aminobenzamide mimics and the Pteridine mimics on prokaryotic and eukaryotic cells is dosage dependent and also depends on the general health-condition of the cells

The unexpectedly increased effectiveness provided by the combined application of sulfadimidine and metronidazole, as it was demonstrated in the granted U.S. Pat. No. 6,432,935, partly can be explained by the antifolic acid effect of the sulfonamides and the simultaneous cytotoxic effect of the nitroimidazoles strengthening each other when are acting together on bacteria or on the weakened ill human cells. The possible further explanation of the increased effectiveness could be that both of them have antitumor effectiveness as it is summarized briefly below: In the 1940s scientists discovered that sulfanilamide blocked the activity of an enzyme called carbonic anhydrase. In 1993, Teicher et al. reported that carbonic anhydrase (CA) inhibitors, as part of a chemotherapy regimen, enhanced the chemotherapeutic drug effects and suppressed the tumor cells. (In 2008. Supuran Claudiu T reported that the carbonic anhydrase inhibitors beside their antitumor effect have antimicrobial effects too because they are targeting CAs from pathogenic organisms such as Helicobacter pylori, Mycobacterium tuberculosis, Plasmodium falciparum, Candida albicans, etc.) The nitroimidazoles also have certain anticancer effects. In 1989 Trujillo J M et al evaluated metronidazole as a single agent or in combination with cis-diamminedichloroplatinum and 1-beta-arabinofuranosylcytosine for its cytotoxic effects on five established human colon carcinoma cell lines. Metronidazole enhanced the synergism resulting from the combination of the two antitumor agents and alone produced dose-dependent cytotoxic effect.

When the 1-(2 hydroxy-ethyl)-2-methyl-4-/(4′amino-phenyl-sulfonyl)amino/-5-nitroimidazole, one of the sulfonamides (or the present sulfonamide or its derivatives or a sulfanilamide molecule respectively substituted similarly to Prontosil but by a nitroimidazole molecule) described below has entered into a cell, then its metabolism occurring at the place of the substitution results in the originally (before substitution) separate two molecules of acting agents (sulfanilamide and nitroimidazole). Application of the present molecules in the combinations of U.S. Pat. No. 6,432,935 provides a very important advantage, namely the simultaneous entering into the cells of two active agents of the combinations. Naturally, this advantage is present when any of the present molecules is applied alone as a monotherapy.

The scope of the antimicrobial including antiviral effectiveness and also the observed antitumor efficiency of the combined compositions remain at least the same in the cases of the present compositions containing the described present molecule(s) instead of one of the known sulfonamides and the nitroimidazole molecule components. The possible theoretical bases and the effectiveness mentioned above have been further examined in Petri dishes.

Direct and Indirect Anti HIV/AIDS Effectiveness. The international recognition of U.S. Pat. No. 6,432,935 was expressed through prizes awarded for the anti HIV/AIDS effectiveness of such pharmaceutical compositions, in Moscow(1), Geneva(2), and Warsaw(3) 1.) VIII. Moscow International Salon of Innovations and Investments (inventions, investment-attractive innovations, high technologies) 3-6 Mar., 2008, All-Russian Exhibition Center, Pavilion 69, Moscow. 2.) 36. International Exhibition of Inventions, New Techniques and Products in Geneva, on 2-6 Apr. 2008. 3.) IWIS—International Warsaw Inventions Show 2008 on Jun. 5, 2008. Palace of Culture & Science.

The importance of the combined treatment for bacterial vaginosis was demonstrated in a comparative, in vivo, human, prospective, single blind, clinical and microbiological diagnoses based and randomised study (see Milankovits, M., Baksay, L., Plachy, J.: Comparative study of combined local treatment (sulfadimidine, metronidazole and nystatin) and the standard monotherapy in uncomplicated bacterial vaginosis. Orv Hetil. 2002 Dec. 22; 143(51):2835-40.) and later presented with accomplished observations (see Milankovits, M.: The effect of combined antimicrobial treatment on prokaryotic cells and on certain groups of eukaryotic cells. Oral presentation. World and Ehrlich Conference, Nürnberg, Sep. 9-11, 2004.) The effectiveness of the presently available and applied monotherapies for bacterial vaginosis is less than optimal (see Erica Weir: Bacterial vaginosis: more questions than answers CMAJ 2004; 171: 448). Only combined treatment can provide optimal efficacy (see Milankovits, M.: More effective treatment for bacterial vaginosis. CMAJ, eLetters for Weir. 4 Mar. 2005). The more effective combined treatment often times prevents the development of chronic diseases (see Milankovits, M.: Infections and chronic diseases. Nov. 11, 2005. Comments on: The neglected epidemic of chronic disease The Lancet—Vol. 366, Number 9496, 29 Oct. 2005, Pages 1514).

An additional aspect and embodiment of the present invention is the application of the new molecules in Photomedicine. The compositions of U.S. Pat. No. 6,432,935 were applied successfully by Sommer. (see Andrei P. Sommer Antiinfectives and Low-Level Light: A New Chapter in Photomedicine Photomedicine and Laser Surgery. Jun. 1, 2007, 25(3): 150-158. doi:10.1089/pho.2007.2058.

The examples, including those having a folic and antagonistic effect and nitromidazole or nitromidazole and fluor substitution, above and below serve to further typify the nature of the invention but should not be construed as a limitation. The following syntheses reference “Compounds”, which are structurally illustrated in the “Drawings” section below.

Synthesis of Nitroimidazole Derivatives

### A. 1,2-Substituted-4-[(4′-amino-phenyl-sulfonyl)amino]-5-nitroimidazoles

The first part of the present pharmaceutically active materials (Compound I, P¹=H) may be easily prepared from substituted 4-amino-5-nitroimidazoles (Compound II). It is well known that protected 4-amino-benzenesulfonyl halogenide Compound II (such as N-acetylsulfanilyl chloride, Compound II, P¹=acetyl; X=Cl) can react with various heteroaryl amines resulting protected N-heteroaryl-4-amino-sulfanyl amides. This reaction was described in several publications e.g. J. Org. Chem. 2264-2267 (1961). After removing the protective group P¹, the desired material Compound I (P¹=H) is obtained.

The necessary intermediate Compound III may be prepared from the corresponding substituted 5-nitroimidazoles (Compound IV) by described or other amination methods. It is known from the literature that 4- or 5-nitroimidazoles may be aminated to the corresponding 5- or 4-amino-nitroimidazoles. This reaction was effected when nitroimidazole was treated with hydroxylamine in an alcoholic solvent (J. Heterocycl. Chem. 6 53-60 (1969)), or with 4-amino-1,2,4-triazole in dimethyl sulfoxide (Tetrahedron 49 5339-5350 (1993)). Any other amination method may also be applicable to prepare Compound III from Compound IV.

Starting 5-nitroimidazoles are commercially available, known materials. E.g. the commercially available 2-methyl-5-nitroimidazole (Compound IV, R=methyl; R═H) may be treated with the appropriate halogenide of R² and Compound IV is obtained. Some materials described by the structure of Compound IV are known drugs. When R¹=methyl and R²=2-hydroxyethyl, Compound IV is the known pharmaceutically active ingredient metronidazole. When R¹=methyl and R²=2-(ethylsulfonyl)-ethyl, Compound IV is tinidazole. Tinidazole can be aminated by the mentioned methods directly. Amination of metronidazole, however, is more advantageous if the hydroxy group is in protected form (Compound V). P¹ and P² groups may be different, e.g. any acyl, benzyl, but it is more economic if these groups may be removed with one reaction step. If both P¹ and P² groups are acyl, e.g. acetyl, these may be removed with simple hydrolysis.

### B. 2,4-Diamino-6-[(N-(2-methyl-5-nitro-1-imidazolylalkyl)-N-phenyl)amino-methyl]-quinazolines

The second part of the pharmaceutically active materials (Compound VIII, P³ and P⁴ are hydrogen) may be prepared from known starting materials. Synthesis of some 2,4-diamino-6-[(N-alkyl-N-phenyl)amino-methyl]-quinazolines were described (J. Heterocyclic Chem. 24 345-349 (1987) where alkyl group was methyl, ethyl, or isopropyl. It was observed that certain 2-alkyl-5-nitroimidazoles substituted at position 1 by a haloalkyl group having at least two carbon atom chain length (Compound IX) can react with similar protected 2,4-diaminoquinazolines (Compound X) forming new imidazolylalkyl derivatives Compound VIII. After removing protective groups P³ and P⁴, the pharmaceutically active materials (Compound VIII, P³ and P⁴ are hydrogen) are obtained.

If in Compound IX Q=bond, R¹=methyl, X=chlorine, the Compound IX may be prepared easily from the mentioned metronidazole (Compound V, P²═H) by chlorination. This reaction and compound are known and were described in more publications (e.g. Arzneim. Forsch. 16 23-25 (1966)).

If in Compound IX Q=—SO₂CH₂—CH₂—, R=methyl, X=Cl, Compound IX may be prepared from commercially available 2-methyl-5-nitroimidazole and known material bis-(2-chloroethyl) sulfone similarly to synthesis of other 1-alkyl-2-methyl-nitroimidazoles (e.g. Synth. Commun. 23 2611-2616 (1993)).

Some of the materials described by Compound X are also known materials. If in Compound X R³=3,4,5-trimethoxy; R⁴═CH₃; P³ and P⁴=H, the Compound X is trimetrexate. Protecting groups P³ and P⁴ are advantageously benzoyl or other groups which may be removed after reaction with Compound IX. Synthesis of benzoyl protected such derivatives were also published in the mentioned publication J. Heterocyclic Chem. 24 345-349 (1987). According to this paper, removing of the P³ and P⁴ groups of Compound 8 is affected by treatment of such compound with methanolic sodium methoxide.

### C. 1,2-Substituted-4-[(4′ sulfonamido-phenyl)amino]-5-nitroimidazoles

The third part of the pharmaceutically active materials (Compound XIII) may be prepared by known synthetic procedures. It is known from the literature that substituted 4,5-dinitroimidazoles can react with substituted anilines to form 4-phenylamino-5-nitroimidazoles (Monatshefte für Chemie 134(8) 1145-1150 (2003)). Such a reaction may be performed with O-protected sulphanilic acid (Compound XIV) and 1- and 2-substituted-4,5-dinitroimidazoles (Compound XV) when Compound XVI is obtained. After removing P⁵ protective group, Compound XIII is obtained.

A part of these pharmaceutically active materials described by Compound XIII when R¹ is methyl. Starting material of these compounds are 3-substituted-2-methyl-4,5-dinitroimidazoles (Compound XV, R¹=methyl). These materials may be prepared by known methods e.g. as described in Pharmazie 44 817-820 (1989)) by nitration and then alkylation of 2-methyl-4-nitroimidazole.

An example of these materials is Compound XVIII. In this case, starting material Compound XV contains R=methyl and R=2-(ethylsulfonyl)-ethyl. The synthesis consist of the following steps: (i) reaction of dinitroimidazole with Compound XIV; (ii) removing of protective group P⁵; (iii) transformation of sulfonic acid to sulfonamide by known procedures.

Other advantageous example of these materials is Compound XIX. In this particular case, starting material Compound XVII contains protective group P². The synthesis consist of the following steps: (i) reaction of dinitroimidazole with Compound XIV; (ii) removing protective group P⁵; (iii) transformation of sulfonic acid to sulfonamide by known methods; (iv) removing of P².

### D. 1,2-Substituted-4-[(4′-fluoro-phenyl-sulfonyl)amino]-5-nitroimidazoles

These pharmaceutically active materials are illustrated by the general formula of Compound XX. These materials may be prepared similarly as it was described above for the analogous 4-amino derivatives (Compound I). In this case, aminonitroimidazoles (Compound III) can be reacted with commercially available 4-fluorosulfanylil halogenides Compound XXI.

If in Compound III R=methyl and R²=2-(ethylsulfonyl)-ethyl, after reaction with Compound XXI, Compound XXII is obtained. If in Compound III R¹=methyl and R²=protected-ethoxy, after reaction with Compound XXI and removing of the O-protective group, Compound XXIII is obtained.

It is important to note that Compound XXII and Compound XXIII are fluorous analogues of Compound VI and Compound VII.

### EXAMPLE 1

### Synthesis of 1-[2-(ethylsulfonyl)-ethyl]-2-methyl-4-[(4′-amino-phenyl-sulfonyl)amino]-5-nitroimidazole (Compound VI)

### 1-[2-ethylsulfonyl)-ethyl]-2-methyl-4-amino-5-nitroimidazole

A mixture of 5.6 g (22.7 mmol) tinidazole (1-[2-(ethylsulfonyl)-ethyl])-2-methyl-5-nitroimidazole), 9.8 g (142 mmol) of hydroxylamine hydrochloride and 200 ml of anhydrous ethanol was stirred and cooled to 5-10° C. At this temperature, 19.5 g (350 mmol) potassium hydroxide in 50 ml of methanol was added drop-wise during 1 hour. The resulting yellow suspension was stirred for 1 hour and then neutralized by concentrated hydrochloric acid to pH 7-8. The precipitate was collected by filtration, washed with water, and dried. The yellow solid (4.8 g, 80%) was pure title compound with structure of Compound III where R¹=methyl, R²=2-(ethylsulfonyl)-ethyl, and has a melting point of 156-158° C.

### 1-[2-(ethylsulfonyl)-ethyl]-2-methyl-4-[(4′-acetylamino-phenyl-sulfonyl)amino]-5-nitroimidazole

Dry N,N-dimethylformamide (50 ml), 4.8 g (18 mmol) 1-[2-ethylsulfonyl)-ethyl]-2-methyl-4-amino-5-nitroimidazole, 4.67 g (20 mmol) 4-acetylsulfanilyl chloride, 3.0 g (22.0 mmol) potassium carbonate were mixed and stirred at 100° C. for 4 hours. The resulting slurry was then cooled to ambient temperature diluted with deionised water (200 ml). The precipitated product was filtered off, dried in vacuum resulting 7.2 g (86%) of expected material as a yellow solid giving a melting point 245° C. (dec.). Proton NMR spectrum was conform to the expected structure.

### 1-[2-(ethylsulfonyl)-ethyl]-2-methyl-4-[(4′-amino-phenyl-sulfonyl)amino]-5-nitroimidazole

A mixture of aqueous sodium hydroxide solution (100 ml, 0.5 M) and 7.2 g (15.7 mmol) of 1-[2-(ethylsulfonyl)-ethyl]-2-methyl-4-[(4′-acetylamino-phenyl-sulfonyl)amino]-5-nitroimidazole was stirred at 60° C. for 5 hours. After cooling, the solid was filtered off, dried in vacuum resulting 5.8 g (89%) yellow solid with melting point 225° C. (dec.). Proton NMR analysis was in agreement of the expected structure.

### EXAMPLE 2

### Synthesis of 1-(2-hydroxy-ethyl)-2-methyl-4-[(4′-amino-phenyl-sulfonyl)amino]-5-nitroimidazole (Compound VII)

### 1-(2-Acetoxy-ethyl)-2-methyl-5-nitroimidazole

A mixture of 10.0 g (58.4 mmol) metronidazole (1-(2-hydroxy-ethyl])-2-methyl-5-nitroimidazole), 50 ml acetic acid, and 15 ml of acetic anhydride was stirred at 60° C. for 3 hours. The acetic acid was distilled off at vacuum, and the residue was stirred with 100 ml of saturated aqueous sodium hydrocarbonate for 30 min. The precipitated solid was filtered off, dried in vacuum resulting 11.8 g (95%) yellow solid which meted at 143-145° C. and had appropriate proton NMR spectrum.

### 1-(2-Acetoxy-ethyl)-2-methyl-4-amino-5-nitroimidazole

To a solution of 11.8 g (55.5 mmol) 1-(2-acetoxy-ethyl)-2-methyl-5-nitroimidazole and 23.3 g (277.5 mmol) of 4-amino-1,2,4-triazole in 90 ml of anhydrous dimenthyl sulfoxide was added at room temperature a suspension of 30.0 g (0.555 mol) of sodium methoxide in 300 ml of anhydrous dimethyl sulfoxide. The red suspension obtained was stirred at room temperature for 2 hours and then poured into 1.5 L of saturated ammonium chloride solution. The precipitated solid was filtered off, rinsed with water, and dried in vacuum. The obtained material was 6.59 g (52%) yellow solid which decomposed at 245° C. The NMR spectrum was in agreement with the expected structure.

### 1-(2-Acetoxy-ethyl)-2-methyl-4-[(4′-acetylamino-phenyl-sulfonyl)amino]-5-nitroimidazole

Dry N,N-dimethylformamide (75 ml), 6.59 g (28.9 mmol) 1-(2-acetoxy-ethyl)-2-methyl-4-amino-5-nitroimidazole, 7.01 g (30 mmol) 4-acetylsulfanilyl chloride, 4.56 g (33.0 mmol) potassium carbonate were mixed and stirred at 100° C. for 5 hours. The resulting slurry was then cooled to ambient temperature diluted with deionised water (300 ml). The precipitated product was filtered off, dried in vacuum resulting 9.6 g (78%) of expected material as a yellow solid giving a melting point of 220° C. (dec.). Proton NMR spectrum was conform to the expected structure.

### 1-(2-Hydroxy-ethyl)-2-methyl-4-[(4′-amino-phenyl-sulfonyl)amino]-5-nitroimidazole

A mixture of aqueous sodium hydroxide solution (100 ml, 0.5 M) and 9.6 g (22.5 mmol) of 1-(2-acetoxy-ethyl)-2-methyl-4-[(4′-acetylamino-phenyl-sulfonyl)amino]-5-nitroimidazole was stirred at 60° C. for 5 hours. After cooling, the solid was filtered off, dried in vacuum resulting 7.0 g (91%) yellow solid with melting point 225° C. (dec.). Proton NMR analysis was in agreement of the expected structure.

### EXAMPLE 3

### Synthesis of 2,4-Diamino-5-methyl-6-[(N-(2-methyl-5-nitro-1-imidazolyl-ethyl)-N-(3,4,5-trimethoxy-phenyl))amino-methyl]-quinazoline (Compound XI)

A mixture of 5.77 g (10.0 mmol) of 2,4-dibenzoylamino-5-methyl-6-[N-(3,4,5-trimethoxy-phenyl)amino-methyl]-quinazoline, 1.90 g (10 mmol) of 1-(2-chloro-ethyl)-2-methyl-5-nitroimidazole, 1.30 g of potassium carbonate and 100 ml of N,N-dimethylformamide were stirred and heated at 100-105° C. for 5 hr. After cooling, the solvent was removed by vacuum distillation, and the residue was treated with 50 ml of hot water. The solid was filtered off, and dried in vacuum at 80° C. until constant weight resulting 7.10 g crude 2,4-dibenzoylamino-5-methyl-6-[(N-(2-methyl-5-nitro-1-imidazolyl-ethyl)-N-(3,4,5-trimethoxy-phenyl))amino-methyl]-quinazoline.

The crude dibenzamide was dissolved in methanol (200 ml), 1.08 g (20 mmol) sodium methoxide was added, and the resulting solution was heated under reflux for 1 hour. Then the solvent was distilled off, and the resulting oily residue was treated with 2×40 ml of 1 N aqueous sodium hydroxide. The solid residue was dried in vacuum at 80° C. Yellow solid material was obtained (2.77 g, 53%) which had a melting point of 182-186° C.

### EXAMPLE 4

### Synthesis of 2,4-diamino-5-methyl-6-[(N-(2-(2-(2-methyl-5-nitro-1-imidazolyl-ethyl)sulfonyl)ethyl)-N-(3,4,5-trimethoxy-phenyl))amino-methyl]-quinazoline (Compound XII)

### 1-(2-(2-chloro-ethyl)sulfonyl)ethyl)-2-methyl-5-nitroimidazole

A mixture of 12.7 g (0.100 mol) of 2-methyl-5-nitroimidazole, 38.2 g (0.200 mol) of bis-(2-chloroethyl) sulfone, 20 g 13.8 g (0.100 mol) potassium carbonate, and 100 ml of acetonitrile was stirred under reflux for 2 hours. Then the solvent was distilled off, and the salts were dissolved with water. The remaining solids were filtered off, dried, and crystallised from acetonitrile resulting 18.3 g (65%) light-yellow solid which melts at 75-77° C.

### 2,4-Dibenzoylamino-5-methyl-6-[(N-(2-(2-(2-methyl-5-nitro-1-imidazolyl-ethyl)sulfonyl)ethyl)-N-(3,4,5-trimethoxy-phenyl))amino-methyl]-quinazoline

A mixture of 5.77 g (10.0 mmol) of 2,4-dibenzoylamino-5-methyl-6-[N-(3,4,5-trimethoxy-phenyl)amino-methyl]-quinazoline, 2.82 g, 1-(2-(2-chloro-ethyl)sulfonyl)ethyl)-2-methyl-5-nitroimidazole 1.30 g of potassium carbonate and 100 ml of N,N-dimethylformamide were stirred and heated at 100-105° C. for 6 hr. After cooling, the solvent was removed by vacuum distillation, and the residue was treated with 50 ml of hot water. The solid was filtered off, and dried in vacuum at 80° C. until constant weight resulting 7.30 g crude dibenzamide.

### 2,4-Diamino-5-methyl-6-[(N-(2-(2-(2-methyl-5-nitro-1-imidazolyl-ethyl)sulfonyl)ethyl)-N-(3,4,5-trimethoxy-phenyl))amino-methyl]-quinazoline

The crude dibenzamide was dissolved in methanol (200 ml), 1.08 g (20 mmol) sodium methoxide was added, and the resulting solution was heated under reflux for 45 min. Then the solvent was distilled off, and the resulting oily residue was treated with 2×40 ml of 1 N aqueous sodium hydroxide. The solid residue was dried in vacuum at 80° C. Yellow solid material was obtained (4.0 g, 65%) which had a melting point of 136-139° C.

### DRAWING

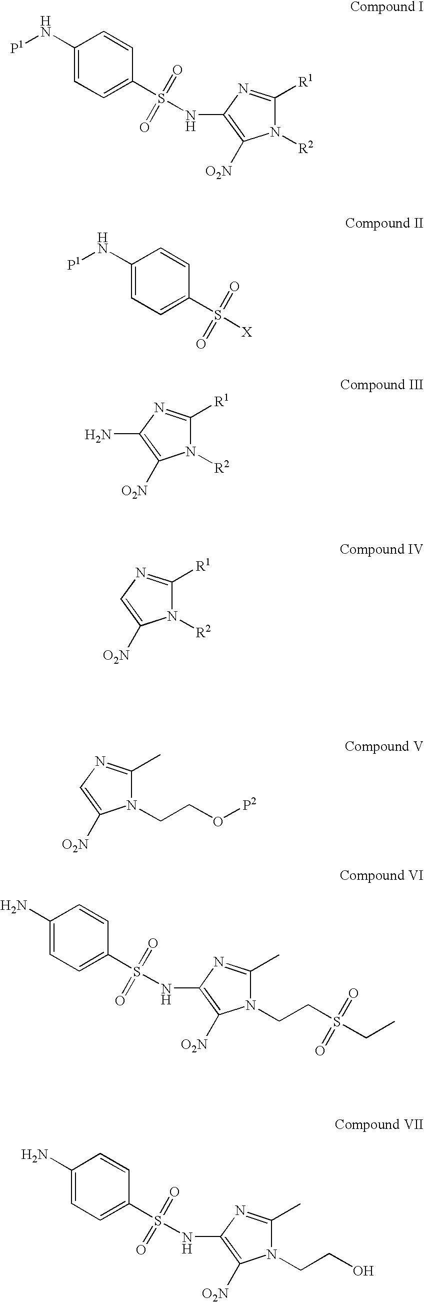

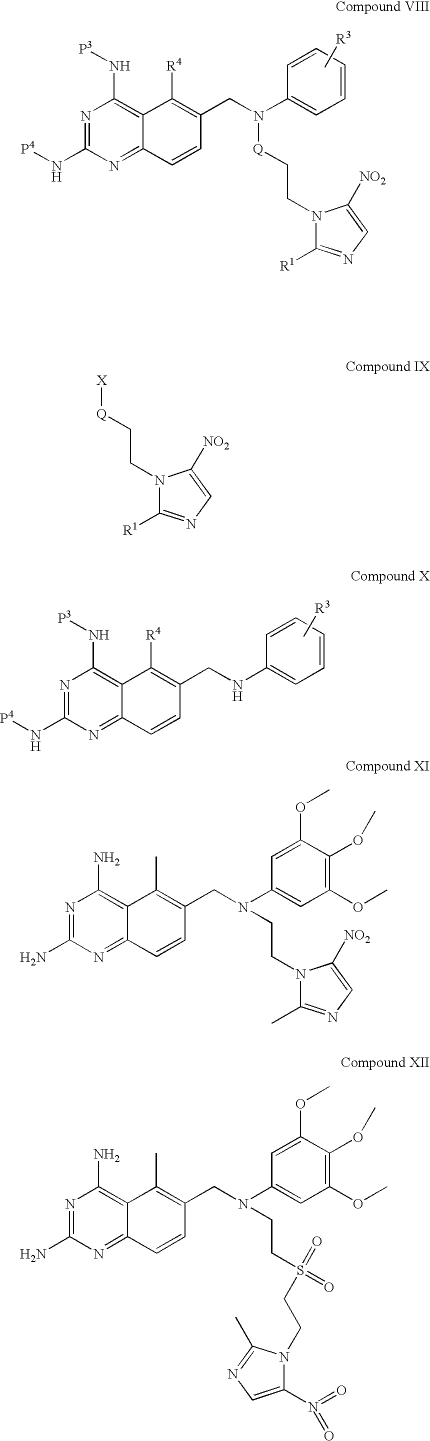

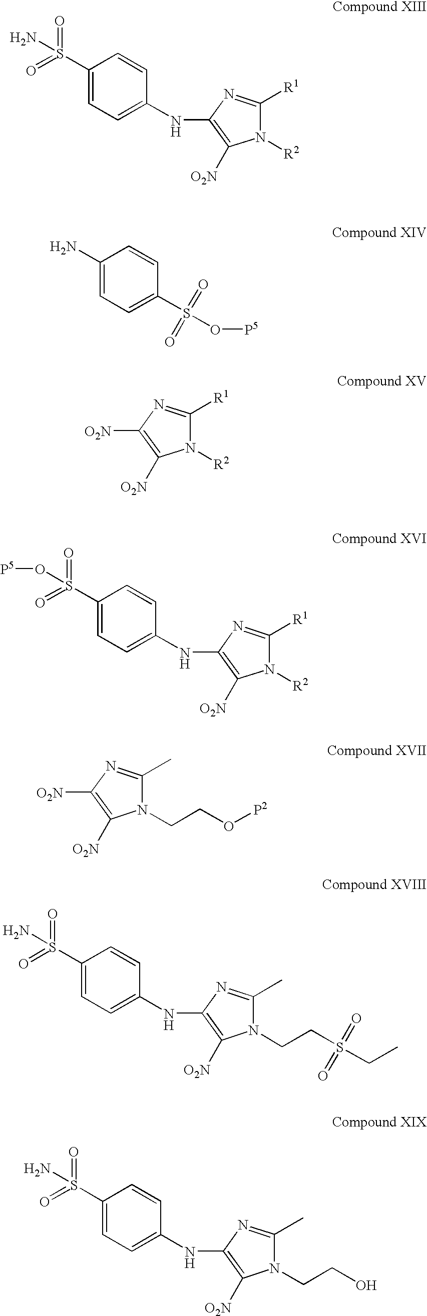

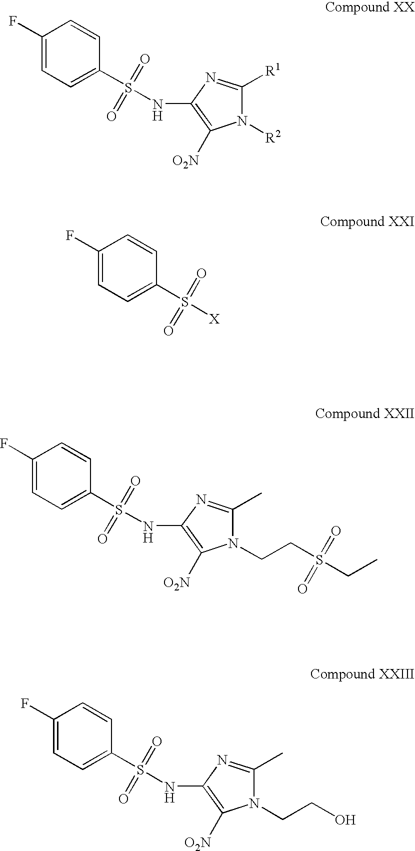

The enhanced antimicrobial activity provided by the combined treatment cures the infection quickly and so the immune stimulus shortens and it results in a weaker immune response. Vaccination against pathogen microbes of the vagina, by vaccines prepared and applied by known techniques, improves the immune response and resistance of the body after the antimicrobial treatment. The combined treatment and vaccination together is especially important for the treatment of infertility (of infectious origin) and dental diseases.

The following examples demonstrate the effectiveness of a pharmaceutical composition containing a sulfonamide and/or a nitroimidazole with and without trimethoprim or clindamycin.

### EXAMPLE 14

11 Patients
Seven days treatment

| | |
|---|---|
| Sulfadimidin: | 0.10 g |
| Metronidazole: | 0.40 g |
| Massa | 2.00 g |
| Polyoxaethenum: | |

Completely treated: 7
Remained the same: 4
Worsened: 0

### EXAMPLE 15

12 Patients
Seven days treatment

| | |
|---|---|
| Sulfadimidin: | 0.10 g |
| Metronidazole: | 0.40 g |
| Natrium | 0.05 g |
| Tetraboratum: | |
| Massa | 2.00 g |
| Polyoxaethenum: | |

Completely treated: 8
Remained the same: 4
Worsened: 0

### EXAMPLE 16

6 Patients
Seven days treatment

| | |
|---|---|
| Sulfamethoxazol: | 0.10 g |
| Trimethoprim: | 0.02 g |
| Metronidazole: | 0.20 g |
| Massa | 2.00 g |
| Polyoxaethenum: | |

Completely treated: 5
Remained the same: 1
Worsened: 0

### EXAMPLE 17

7 Patients
Seven days treatment

| | |
|---|---|
| Clindamycin: | 0.05 g |
| Sulfadimidin: | 0.10 g |
| Massa | 2.00 g |
| Polyoxaethenum: | |

Completely treated: 4
Remained the same: 3
Worsened: 0

### EXAMPLE 18

7 Patients
Seven days treatment

| | |
|---|---|
| Sulfadimidin: | .10 g |
| Metronidazole: | .20 g |
| Nystatin: | .10 g |
| Natamycin: | .20 g |
| Massa | 2.00 g |
| Polyoxaethenum: | |

Completely treated: 6
Remained the same: 1
Worsened: 0

Before treatment using the present combinations, vaginal bacterial samples were taken with cotton swabs (for culture and gram stain). The most common microorganisms found were E. Coli, Enterococcus faecalis, B-group streptococci, Candida albicans, Urea plasma urealyticum, Mycoplasma hominis, Gardnerella, Trichomonas vaginalis, Chlamydia trachomatis, and, to a lesser extent, Staphylococci, Proteus, Klebsiella, Haemophylus, etc. Antibiotic sensitivity was examined as well. After taking samples, patients were treated with the above-listed combinations of the present invention.

In addition to providing an unexpected increase in efficacy of each individual component when utilized in the combinations of the present invention, a significantly lower amount of each active ingredient is needed in the present invention as compared to typical monotherapy formulations.

The effective treatment of bacterial vaginosis is especially important because of its serious clinical implications and morbidity such as post-hysterectomy vaginal cuff cellulitis, plasmacell endometritis. In pregnant women, such clinical implications include amniotic fluid infection, clinical chorioamnionitis, postpartum endometritis, premature rupture of the membranes, pre-term delivery, and low birth weight.

The compositions of the present invention further displayed unexpected anti-viral activity. For example, in cases where the treatment of Condyloma acuminatum (caused by the human papilloma virus) failed, treatment using the compositions of the present invention along with local Podophyllin treatment proved to be permanently successful in 66% of the cases treated. Moreover, it has been observed that patients with frequently recurring herpes genitalis became permanently free of symptoms following treatment with the preparations of the present invention in 75% of the cases tested.

The following additional examples are contemplated by the present invention for use in treatment compositions:

Combination I:

| | |
|---|---|
| Chloramphenicol | 0.08 g |
| Sulfadimidin | 0.20 g |
| Clotrimazol | 0.15 g |
| Metronidazole | 0.40 g |
| Massa polyoxaethenum | 2.0 g |

Combination II:

| | |
|---|---|
| Chloramphenicol | 0.1 g |
| Sulfadimidin | 0.1 g |
| Clotrimazol | 0.1 g |
| Metronidazole | 0.40 g |
| Massa polyoxaethenum | 2.0 g |

Combination III:

| | |
|---|---|
| Unasyn | 0.1 g |
| Sulphadimidin | 0.1 g |
| Nystatin | 0.1 g |
| Metronidazole | 0.4 g |
| Massa polyoxaethenum | 2.0 g |

Combination IV:

| | |
|---|---|
| Augmentin | 0.1 g |
| Sulphadimidin | 0.1 g |
| Nystatin | 0.1 g |
| Natrium-tetraborat | 0.05 g |
| Metronidazole | 0.4 g |
| Massa polyoxaethenum | 2.0 g |

Combination V:

| | |
|---|---|
| Oxacillin | 0.1 g |
| Sulphadimidin | 0.1 g |
| Nystatin | 0.1 g |
| Metronidazole | 0.4 g |
| Massa polyoxaethenum | 2.0 g |

Combination VI:

| | |
|---|---|
| Cefaclor (Ceclor) | 0.05 g |
| Sulphadimidin | 0.1 g |
| Nystatin | 0.1 g |
| Metronidazole | 0.4 g |
| Massa polyoxaethenum | 2.0 g |

Combination VII:

| | |
|---|---|
| Gentamicin | 0.05 g |
| Sulphadimidin | 0.1 g |
| Nystatin | 0.1 g |
| Metronidazole | 0.4 g |
| Massa polyoxaethenum | 2.0 g |

Combination VIII:

| | |
|---|---|
| Clarithromycin (Klacid) | 0.1 g |
| Sulphadimidin | 0.1 g |
| Nystatin | 0.1 g |
| Metronidazole | 0.4 g |
| Massa polyoxaethenum | 2.0 g |

Combination IX:

| | | |
|---|---|---|
| Chloramphenicol | 0.1 | g |
| Sulphadimidin | 0.1 | g |
| Nystatin | 0.1 | g |
| Natrium-tetraborat | 0.05-0.10 | g |
| Metronidazole | 0.4 | g |
| Massa polyoxaethenum | 2.0 | g |

Combination X:

| | |
|---|---|
| Clindamycin | 0.05 g |
| Natrium-tetraborat | 0.05 g |
| Sulphadimidin | 0.1 g |
| Nystatin | 0.1 g |
| Metronidazole | 0.4 g |
| Massa polyoxaethenum | 2.0 g |

The present combination can be used in applications such as suppositories, ointments, talc powder, solution, painting solutions, vaginal drops, or impregnated tampons.

For systemic treatment situations, the preparations contain the combination of the active ingredients in the same ratio as described above for localized treatment.

Ointment preparations contain the combination of the active ingredients in the same ratio as in the vaginal suppository, together with ointment base, with yellow Vaseline and other components known per se, if required. This preparation is especially effective in case of tissue damage (diabetes mellitus, burning, etc.). The talc powder preparation contains the active ingredient combination in solid form, with carriers such as talc, etc. The painting solutions and vaginal drops are prepared with an appropriate organic solvent. The vaginal drop solutions are useful in pediatric gynecology; but can also be used for adults in adequate doses.

The present compositions can also optionally contain borax (NA₂B₄O₇.4H₂O).

The pharmaceutical compositions mentioned above can be prepared by known techniques used in the preparation of the pharmaceutical compositions.

What is claimed is:

1. A pharmaceutical composition, comprising:
(a) one or more antibacterial agents;
(b) one or more antifungal agents effective against a Candida species; and
(c) a nitroimidazole, wherein said one or more antibacterial agents, said one or more antifungal agents, and said nitroimidazole are present in synergistic effective amounts.

2. A pharmaceutical composition as in claim 1, further comprising a pharmaceutically acceptable carrier.

3. A pharmaceutical composition as in claim 1 wherein said one or more antibacterial agents include a sulfonamide.

4. A pharmaceutical composition as in claim 1, further comprising borax.

5. A pharmaceutical composition as in claim 1 wherein said composition is selected from the group consisting of vaginal suppositories, ointments, solutions, painting solutions, vaginal drops, powders and impregnated tampons.

6. A pharmaceutical composition as in claim 1 wherein said one or more antibacterial agents are selected from the group consisting of chloramphenicol, erythromycin, oxytetracyclin, ampicillin, ciprofloxacin, neomycin, polymyxin, unasyn, augmentin, oxycillin, cefaclor, gentamycin, clarithromycin, clindamycin and sulfadimidin.

7. A pharmaceutical composition as in claim 1 wherein said antifungal agent is selected from the group consisting of clotrimazol, natamycin and nystatin.

8. A pharmaceutical composition as in claim 1 being particularly adapted for use in systemic treatment of ascending infections.

9. A pharmaceutical composition as in claim 1 comprising:
(a) 0.04-0.30 grams of said one or more antibacterial agents;
(b) 0.025-0.30 grams of said antifungal agent; and
(c) 0.10-0.45 grams of said nitroimidazole.

10. A pharmaceutical composition consisting essentially of:
(a) an antibacterial agent;
(b) an antifungal agent effective against a Candida species; a nitroimidazole; and
(c) a pharmaceutically acceptable carrier, wherein said antibacterial agent, said antifungal agent and said nitroimidazole are present in synergistic effective amounts.

11. A pharmaceutical composition as in claim 10 wherein said antibacterial agent is a sulfonamide.

12. A pharmaceutical composition as in claim 10 wherein said composition is selected from the group consisting of vaginal suppositories, ointments, solutions, painting solutions vaginal drops, powders and impregnated tampons.

13. A pharmaceutical composition as in claim 10 wherein said antibacterial agent is selected from the group consisting of chloramphenicol, erythromycin, oxytetracyclin, ampicillin, ciprofloxacin, neomycin, polymyxin, unasyn, augmentin, oxycillin, cefaclor, gentamycin, clarithromycin, clindamycin and sulfadimidin.

14. A pharmaceutical composition as in claim 10 wherein said antifungal agent is selected from the group consisting of clotrimazol, natamycin and nystatin.

15. A pharmaceutical composition as in claim 10 being particularly adapted for use in systemic treatment of ascending infections.

16. A pharmaceutical composition consisting essentially of:
(a) a first antibacterial agent;
(b) a sulfonamide;
(c) an antifungal agent effective against a Candida species;
(d) a nitroimidazole; and
(e) a pharmaceutically acceptable carrier, wherein said antibacterial agent, said sulfonamide, said antifungal agent and said nitroimidazole are present in synergistic effective amounts.

17. A pharmaceutical composition, comprising:
(a) one or more antibacterial agents;
(b) one or more antifungal agents effective against a Candida species; and
(c) one or more additional compounds having a folic acid antagonist effect and a nitroimidazole or nitroimidazole and fluor substitution, wherein said one or more antibacterial agents, said one or more antifungal agents, and said one or more additional compounds are present in synergistic effective amounts.

18. A pharmaceutical composition as in claim 17 further comprising borax.

19. A pharmaceutical composition as in claim 17 wherein said one or more additional compounds include:
(a) at least one of nitroimidazole or nitroimidazole and fluor substituted p-Aminobenzamide mimics; or
(b) at least one of nitroimidazole substituted Pteridine mimics.

20. A pharmaceutical composition as in claim 19 further comprising borax.

21. A pharmaceutical composition as in claim 19 wherein said one or more additional compounds include 1-(2 hydroxy-ethyl)-2-methyl-4-/(4′amino-phenyl-sulfonyl)amino/-5-nitroimidazole and/or its derivatives.

22. A pharmaceutical composition as in claim 21 further comprising borax.

23. A pharmaceutical composition as in claim 19 wherein said nitromidazole substituted pteridine mimics include methotrexate.
